(19) United States
(12) Patent Application Publication
(10) Pub. No.: US 2013/0005676 A1
(43) Pub. Date: Jan. 3, 2013

(54) ENHANCING THE THERAPEUTIC EFFECT OF ACUPUNCTURE WITH ADENOSINE

(75) Inventor: Maiken Nedergaard, Webster, NY (US)

(73) Assignee: UNIVERSITY OF ROCHESTER, Rochester, NY (US)

(21) Appl. No.: 13/511,801

(22) PCT Filed: Nov. 24, 2010
(86) PCT No.: PCT/US10/58039

§ 371 Date: Sep. 18, 2012

Related U.S. Application Data
(60) Provisional application No. 61/264,130, filed on Nov. 24, 2009.

(51) Int. Cl. A61K 31/7076; A61K 31/7056; A61P 19/02; A61P 25/00; A61P 29/00

(52) U.S. Cl. 514/46; 514/43

(57) ABSTRACT

The present invention relates to a method of improving the therapeutic effect of acupuncture in a subject. The method involves administering adenosine, an adenosine mimetric, an adenosine modulator, an adenosine transport inhibitor, enzymes involved in adenosine metabolism, and/or an adenosine receptor agonist to the subject under conditions effective to improve the therapeutic effect of the acupuncture.

This application claims benefit of U.S. Provisional Patent Application Ser. No. 61/264,130, filed Nov. 24, 2009, which is hereby incorporated by reference in its entirety.

The subject matter of this application was made with support from the United States Government under The National Institutes of Health, Grant No. NS050315. The government has certain rights in this invention.

### FIELD OF THE INVENTION

The present invention is directed to enhancing the therapeutic effect of acupuncture with adenosine.

### BACKGROUND OF THE INVENTION

Acupuncture is a procedure in which fine needles are inserted and manipulated in patients to relieve pain and other diseases. Acupuncture originates from philosophy-based Eastern medicine and is founded on the concept the vital energy that animates life flows through meridians in the body. Various diseases and conditions will obstruct the flow of energy leading to an undesirable state of unbalance. Acupuncture applied to specific points positioned along the meridians frees the stagnation of energy and restores health. Acupuncture originated in China around 2000 BC and is now in use worldwide (Ernst et al., “Prospective Studies of the Safety of Acupuncture: A Systematic Review,” Am. J. Med. 110:481-485 (2001)). Western Medicine did not unexpectedly meet acupuncture with considerable skepticism (Culliton, B. J., “Acupuncture: Fertile Ground for Faddists and Serious NIH Research,” Science 177:592-594 (1972)). However, as an example of its general acceptance, the Internal Revenue Service listed acupuncture as a deductible medical expense in 1973 and the World Health Organization (WHO) endorses acupuncture for two dozen conditions (Akerele, O., “WHO's Traditional Medicine Programme: Progress and Perspectives,” WHO Chron. 38:76-81(1984)).

Although the analgesic effect of acupuncture is well documented, surprisingly little is known with regard to its biological basis (Lin et al., “Acupuncture Analgesia: A Review of its Mechanisms of Actions,” Am. J. Chin. Med. 36:635-645 (2008)). It is empirically recognized that insertion of the acupuncture needles in itself is not sufficient to relieve pain. An acupuncture session typically last 30 min, during which the needles are intermittently rotated, or electrical stimulation and in some cases heat is applied (Zhao, Z. Q., “Neural Mechanism Underlying Acupuncture Analgesia,” Prog. Neurobiol. 85:355-375 (2008)). The pain threshold is reported to slowly increase and outlast the treatment (Zhao, Z. Q., “Neural Mechanism Underlying Acupuncture Analgesia,” Prog. Neurobiol. 85:355-375 (2008)). The primary mechanism so far implicated in the analgesic effect of acupuncture involves release of opioid peptides in CNS in response to the long-lasting activation of ascending tracks during the intermittent stimulation (Han, J. S., “Acupuncture and Endorphins,” Neurosci. Lett. 361:258-261 (2004); Huang et al., “Characteristics of Electroacupuncture-Induced Analgesia in Mice: Variation with Strain, Frequency, Intensity and Opioid Involvement,” Brain Res. 945:20-25 (2002); Zhao, Z. Q., “Neural Mechanism Underlying Acupuncture Analgesia,” Prog. Neurobiol. 85:355-375 (2008)). However, a centrally acting agent cannot explain why acupuncture conventionally is applied in close proximity to the locus of pain and that the analgesic effects of acupuncture is restricted to the ipsilateral side (Lao et al., “A Parametric Study of Electroacupuncture on Persistent Hyperalgesia and Fos Protein Expression in Rats,” Brain Res. 1020:18-29 (2004); Li et al., “Analgesic Effect of Electroacupuncture on Complete Freund's Adjuvant-Induced Inflammatory Pain in Mice: A Model of Antipain Treatment by Acupuncture in Mice,” Jpn. J. Physiol. 55:339-344 (2005); Zhang et al., “Electroacupuncture Combined With Indomethacin Enhances Antihyperalgesia in Inflammatory Rats,” Pharmacol. Biochem. Behay. 78:793-797 (2004)).

The present invention is directed to enhancing the therapeutic effect of acupuncture.

### SUMMARY OF THE INVENTION

One aspect of the present invention relates to a method of improving the therapeutic effect of acupuncture in a subject. The method involves administering adenosine, an adenosine mimetic, an adenosine modulator, an adenosine transport inhibitor, enzymes involved in adenosine metabolism, and/or an adenosine receptor agonist to the subject under conditions effective to improve the therapeutic effect of the acupuncture.

### BRIEF DESCRIPTION OF THE DRAWINGS

FIGS. 1A-C show that acupuncture triggers release of ATP, ADP, AMP, IMP, inosine, and adenosine. FIG. 1A is a histogram summarizing the mean concentrations of ATP, ADP, AMP, and adenosine during baseline none-stimulated conditions (p=0.125; one way ANOVA, n=8) collected by a microdialysis probe implanted in close proximity to the Zusanli point. FIG. 1B is a representative HPLC chromatogram before (−30 to 0 min), during (0 to 30 min), and after acupuncture (30 to 60 min). Standards are displayed on top. FIG. 1C is a time course of purine release in response to acupuncture (*; p<0.05, **; p<0.01, Tukey-Kramer test compared to −30 min, n=4).

FIGS. 2A-G show anti-nociceptive and anti-hyperalgesic effects of adenosine A1 receptors. FIG. 2A is a schematic of experimental setup. Complete

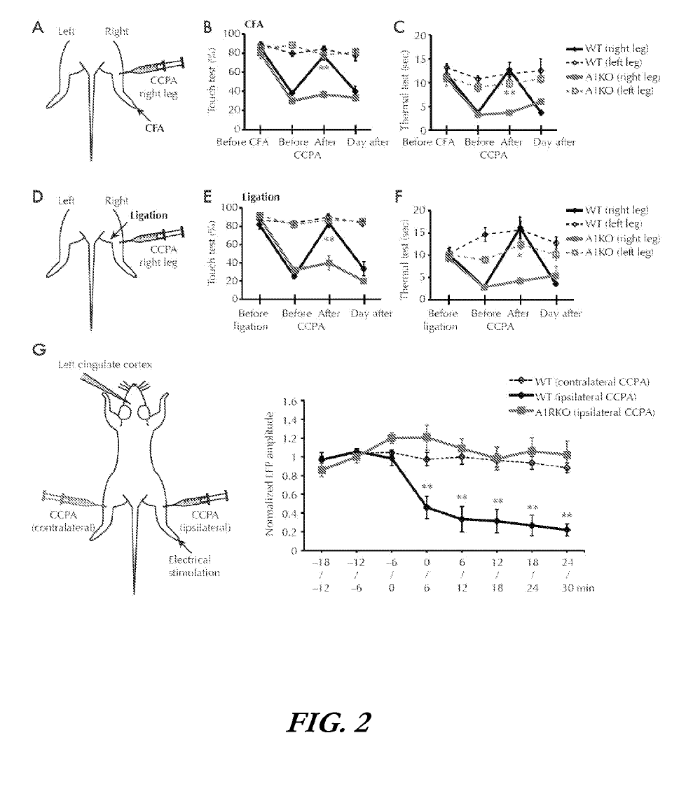

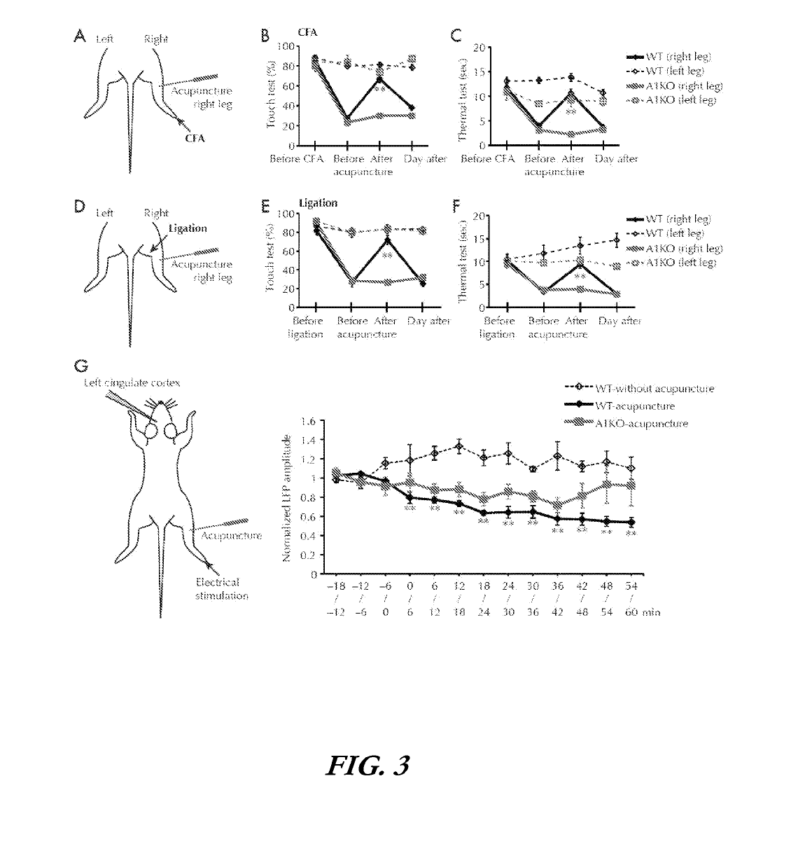

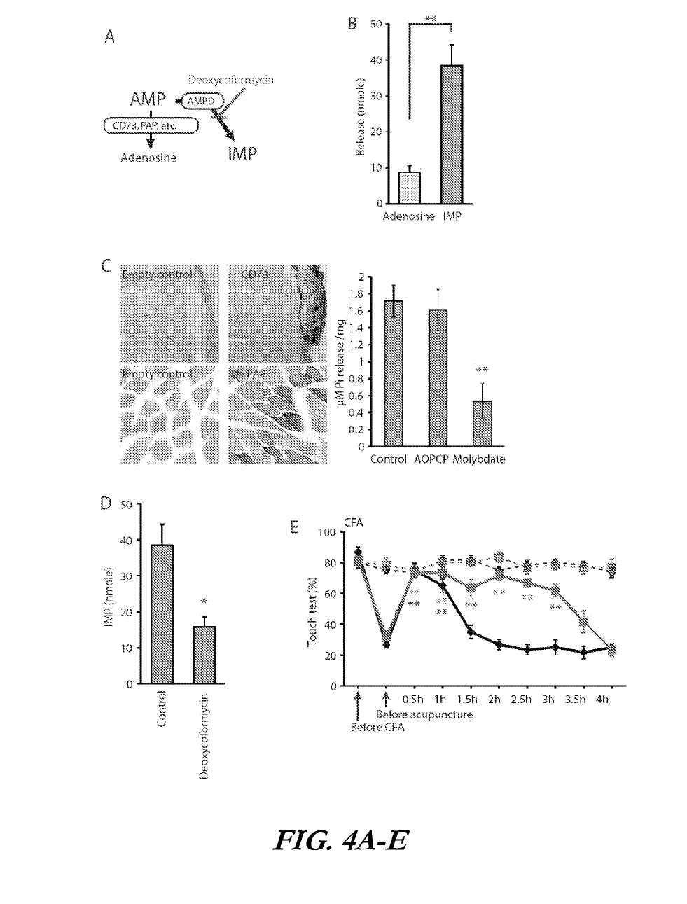

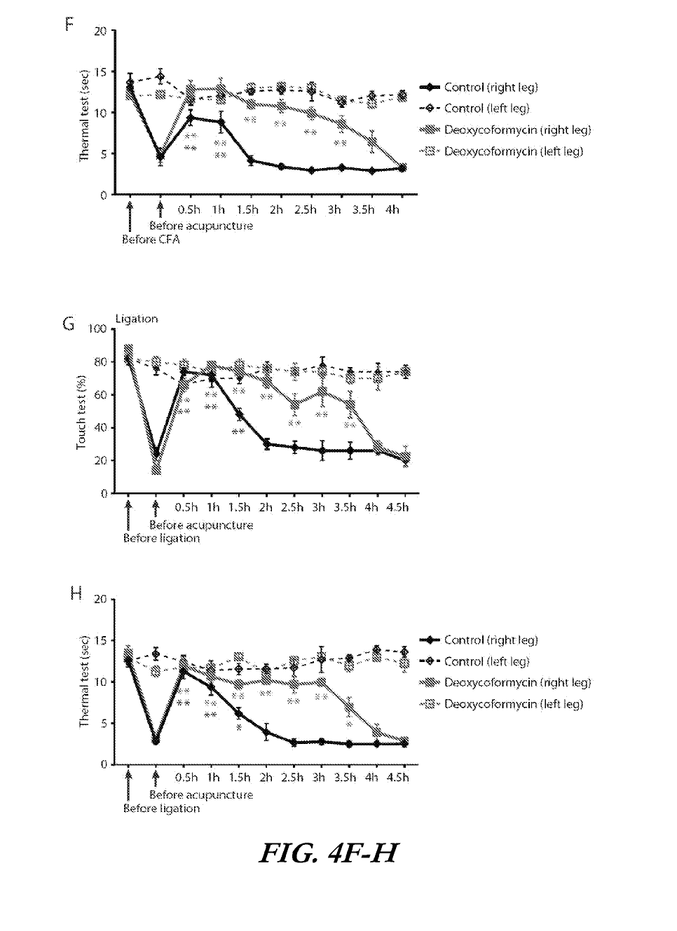

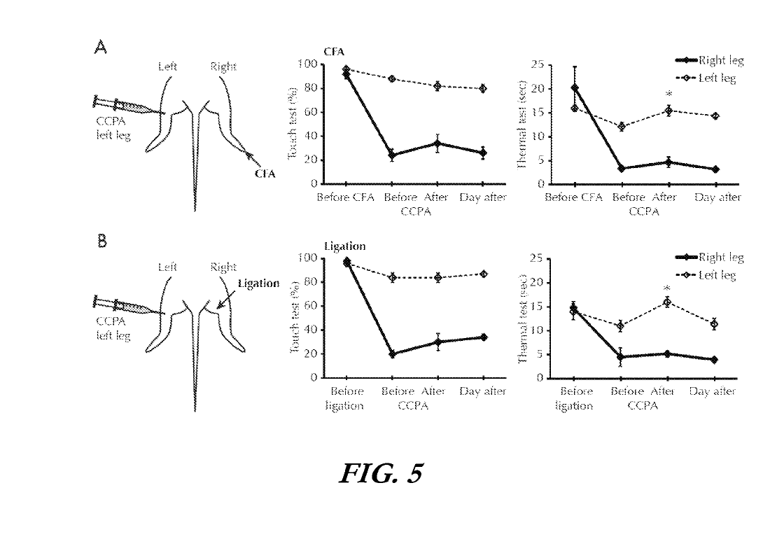

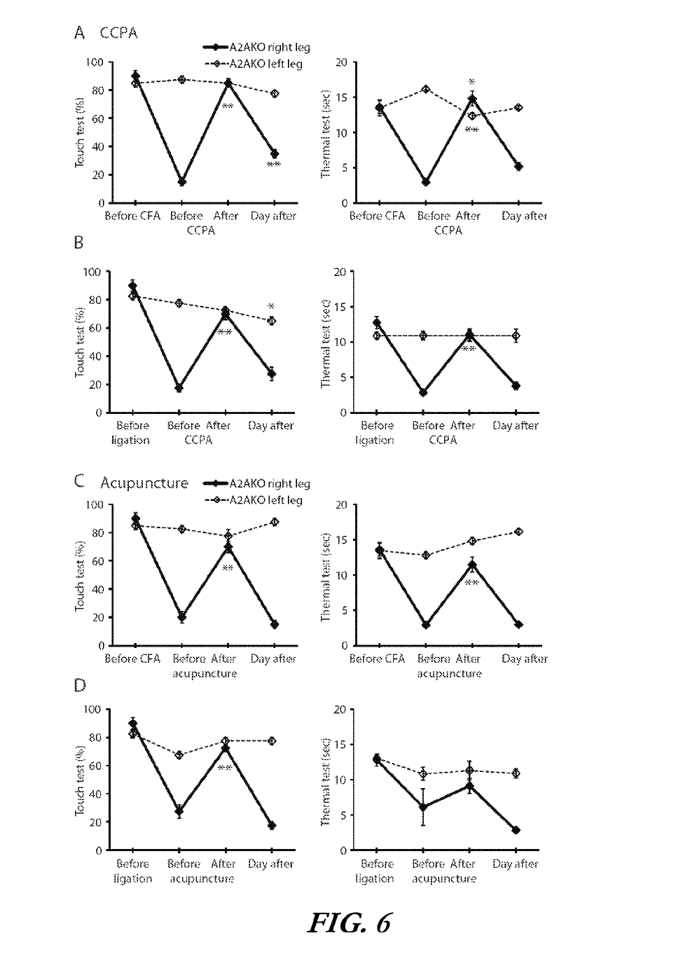

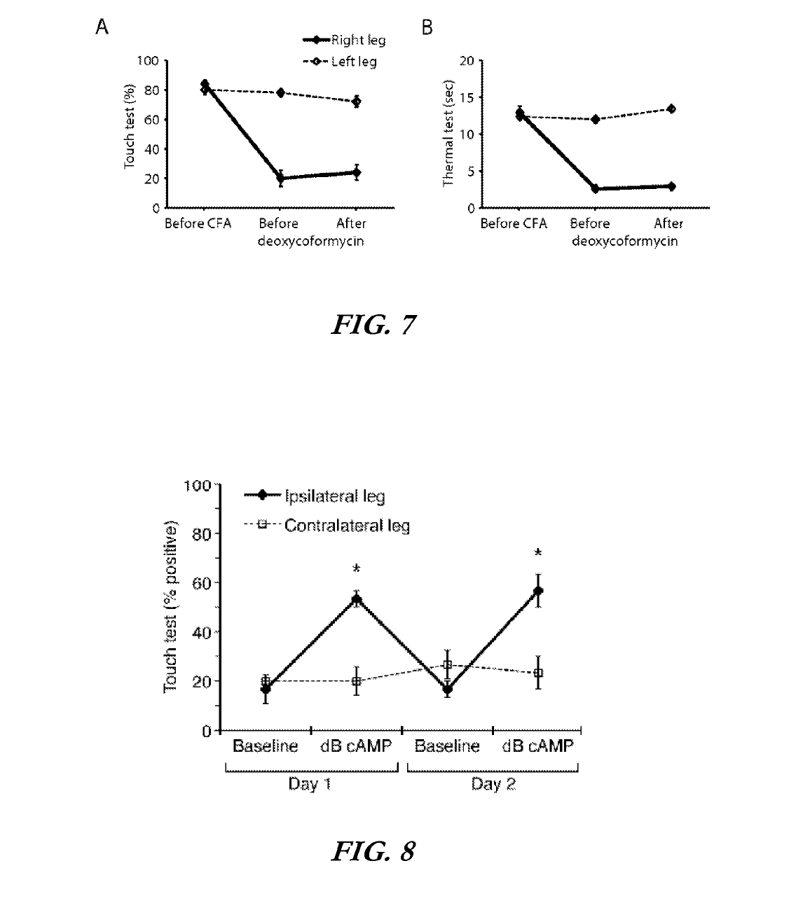

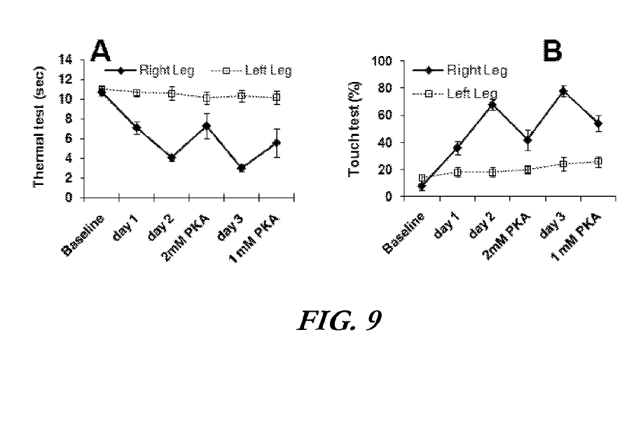

Freund's adjuvant (CFA) was administered in the right paw at day 0. The adenosine receptor agonist, 2-chloro-N(6)-cyclopentyladenosine (CCPA) was injected in the right Zusanli point (ST36) at day 4. FIG. 2B shows a comparison of the effect of CCPA on mechanical allodynia, and FIG. 2C shows the thermal hyperalgesia in wildtype (WT, black) and A1 receptor knockout (A1R KO, red) mice. (**; p<0.01, Tukey-Kramer test compared to before CCPA, n=3-14). FIG. 2D shows that neuropathic pain was evoked by partial ligation of the ischias nerve at day 0 and CCPA administered at day 6. FIG. 2E shows the effect of CCPA on mechanical, and FIG. 2F shows the thermal hypersensitivity in WT and A1R KO mice with ligation of the ischias nerve (*; p<0.05, **; p<0.01, Tukey-Kramer test compared to before CCPA, n=6). FIG. 2G shows the experimental setup used for recording EPSP in left anterior cingulate cortex evoked by painful stimulation of the right foot. The effect of injection of CCPA (0.1 mM, 20 μl) injected in the right or the left Zusanli point on the amplitude of evoked EPSP is plotted as a function of time in WT and A1R KO mice (**; p<0.01, Tukey-Kramer test compared to −18 min, n=4-12).

FIGS. 3A-G show that acupuncture fails to suppress pain in mice lacking adenosine A1 receptors. FIG. 3A is a schematic of experimental layout evaluating the role of A1 receptors in acupuncture-mediated suppression of hyperalgesia in a model inflammatory pain (CFA injected in right paw). FIG. 3B shows that acupuncture reduced sensitivity to both mechanical, and FIG. 3C shows thermal stimulation in WT mice suffering from inflammatory pain after injection of CFA, but not in AIR KO littermates tested at day 4 (**; p<0.01, Tukey-Kramer test compared to before acupuncture, n=3-16). FIG. 3D demonstrates that neuropathic pain was induced by partial ligation of the ischias nerve and the clinical effect of acupuncture tested at day 6. FIG. 3E shows that acupuncture suppressed thermal mechanical allodynia, and FIG. 3F shows that hyperalgesia in WT animal suffering from neuropathic pain, but not in A1R KO mice (**; p<0.01, Tukey-Kramer test compared to before acupuncture, n=6). FIG. 3G shows the experimental setup used to assess the effect of acupuncture on the amplitude of eEPSP in the anterior cingulate cortex evoked by painful foot shock. The amplitude of eEPSP is plotted as a function of time in WT and A1R KO mice (**; p<0.01, Tukey-Kramer test compared to −18 min, n=3-8).

FIGS. 4A-H show that inhibition of AMP deaminase prolongs acupuncture-induced pain relief. FIG. 4A is a schematic diagram outlining the two major pathways of extracellular enzymatic degradation of AMP. FIG. 4B is a histogram comparing the production of adenosine and IMP after incubating tissue sections harvested close to the Zusanli point in 1 mM AMP for 45 min (p=0.008, t-test). FIG. 4C shows the effect of the CD73 inhibitor (AOPCP, 500 μM) and the PAP inhibitor (Molybdate, 500 μM) on phosphate production after incubating tissue harvested close to the Zusanli point in 1 mM AMP for 45 min (*; p<0.05, Tukey-Kramer test compared to control, n=3-8). FIG. 4D shows an inhibitor of AMP deaminase, deoxycoformycin (DCF, 200 μM) reduced production of IMP by ˜50% when tissue harvested close to the Zusanli point was incubated in 1 mM AMP for 45 min (p=0.024, t-test). FIG. 4E shows that DCF prolonged the anti-analgesic effect of acupuncture in WT mice suffering from inflammatory pain to mechanical stimulation and, FIG. 4F to thermal stimulation (*; p<0.05, **; p<0.01, Tukey-Kramer test compared to before acupuncture, n=3-7). FIG. 4G shows that DCF prolonged the beneficial effects of acupuncture in WT mice suffering from neuropathic pain induced by partial ligation of the ischias nerve to mechanical stimulation, and FIG. 4H, to thermal stimulation (*; p<0.05, **; p<0.01, Tukey-Kramer test compared to before acupuncture, n=5).

FIGS. 5A-B show that injection of the A1 receptor antagonists, CCPA in the left leg, contralateral to locus of inflammatory or neuropathic pain has no effect on mechanical and thermal hypersensitivity. FIG. 5A shows that CCPA (0.1 mM, 20 μl) injected in the left Zusanli point had no effect on mechanical and thermal hypersensitivity evoked by inflammatory pain in the right foot. FIG. 5B shows that CCPA injected in the left Zusanli point had no effect on mechanical and thermal hypersensitivity evoked by neuropathic pain in the right foot. (*; p<0.05, Tukey-Kramer test compared to before CCPA, n=5).

FIGS. 6A-D show that mice with deletion of A2a receptors exhibit, similar to WT mice, reduce sensitivity to pain following injection of CCPA or acupuncture. FIG. 6A shows the effect of CCPA (0.1 mM, 20 μl) injected in the right Zusanli point on mechanical and thermal hypersensitivity in A2a receptor KO mice with inflammatory pain. FIG. 6B shows the effect of CCPA on mechanical and thermal hypersensitivity in A2a receptor KO mice with neuropathic pain. FIG. 6C shows the effect of acupuncture on mechanical and thermal hypersensitivity in A2a receptor KO mice with inflammatory pain. FIG. 6D shows the effect of acupuncture on mechanical and thermal hypersensitivity in A2a receptor KO mice with neuropathic pain (*; p<0.05, **; p<0.01, Tukey-Kramer test compared to before CCPA or before acupuncture, n=4).

FIGS. 7A-B shows that deoxycoformycin does not alter the pain sensitivity in the absence of acupuncture. FIG. 7A shows the mechanical hypersensitivity evoked by CFA injection before and after administration of deoxycoformycin (DCF). FIG. 7B shows the thermal hypersensitivity evoked by CFA injection before and after administration of DCF (n=4-6).

FIG. 8 shows that cAMP enhances nociceptive effects in mice. Using Von Frey Filaments, a baseline for pain threshold was established in the wild type. The mice were then injected with 5 mM of dibut 1-cAMP (db-cAMP) in the zunsanli accu-point. To ensure the proper spread of db cAMP, the mice were tested for pain one hour after injections and assessed for touch sensitivity. The experiment was repeated in the same animals 24 h later and yielded similar results.

FIGS. 9A-B show that PKA inhibition reduces nociceptive effects in mice. FIG. 9A shows the thermal sensitivity evoked at baseline and upon injection of PKA inhibitor H-89. FIG. 9B shows the touch sensitivity evoked at baseline and upon injection of PKA inhibitor H-89. Injection of the PKA inhibitor H-89 in the Zusanli points transiently and significantly reduced CFA-induced mechanical pain hypersensitivity in the ipsilateral (right leg), but not the contralateral paw.

### DETAILED DESCRIPTION OF THE INVENTION

The present invention relates to a method of improving the therapeutic effect of acupuncture in a subject. The method involves administering adenosine, an adenosine mimetric, an adenosine modulator, an adenosine transport inhibitor, enzymes involved in adenosine metabolism, and/or an adenosine receptor agonist to the subject under conditions effective to improve the therapeutic effect of the acupuncture.

The method according to the present invention is directed toward improving the therapeutic effect of acupuncture. This therapeutic effect includes, but is not limited to, pain relief and treatment of an inflammatory condition. Examples of the inflammatory condition improved according to the method of the present invention include, without limitation, arthritis and tendinitis.

Administration may be carried out systemically or near the location of the pain or inflammatory condition.

The subjects whose therapeutic effect is improved according to the method of the present invention include, without limitation, humans, monkeys, mice, rats, guinea pigs, cows, sheep, horses, pigs, dogs, and cats.

In one embodiment of the present invention, the administering step involves administration of a protein.

In another embodiment of the present invention, the administering step involves administration of a nucleic acid. Preferably, this is carried out by administering a nucleic acid construct in a viral vector. Examples of suitable viral vectors include an adenoviral vector, a lentiviral vector, a retroviral vector, an adeno-associated viral vector, or a combination thereof The nucleic acid construct includes a promoter, such as a constitutive promoter, a cell-specific promoter, or an inducible or conditional promotor.

Suitable adenosine receptor agonists are adenosine receptor congeners (Jacobson, et al., “Molecular Probes for Extracellular Adenosine Receptors,” Biochem. Pharmacol. 36:1697-1707 (1987); Jacobson, et al. Biochem. Biophys. Res. Commun. 136:1097 (1986); Jacobson, et al., “Adenosine Analogs with Covalently Attached Lipids have Enhanced Potency at Al Adenosine receptors,” FEBS Lett. 225:97-102 (1987), which are hereby incorporated by reference in their entirety), N6-cyclopentyladenosine (Lohse, et al., “2-Chloro-N6-cyclopentyladenosine”: A Highly Selective Agonist at Al Adenosine Receptors,” Naunyn Schmiedebergs Arch. Pharmacol. 337:687-689 (1988); Klotz, et al., “2-Chloro-N6-[3H]cyclopentyladenosine ([3H]CPPA)—A High Affinity Agonist Radioligand for Al Adenosine Receptors,” Naunyn Schmiedebergs Arch. Pharmacol. 340:679-683 (1989), which are hereby incorporated by reference in their entirety); N6-cyclohexyladenosine (Daisley, J. N., et al., Brain Res. 847: 149 (1999); Fraser, H. Br. J. Pharmacol. 128:197 (1999), which are hereby incorporated by reference in their entirety); 2-chloro-cyclopentyladenosine (Klotz, K. N. et al. Naunyn Schmiedebergs Arch. Pharmacol. 340:679 (1989); Lohse, M. J. et al. Naunyn Schmiedebergs Arch. Pharmacol. 337:687 (1988), which are hereby incorporated by reference in their entirety); N-(3(R))-tetrahydrofuranyl)-6-aminopurine riboside (Abstracts From Purines 2000: Biochemical, Pharmacological, and Clinical Perspectives; Conference: Purines 2000: Biochemical, Pharmacological, and Clinical Perspectives, Complutense University of Madrid—Madrid (Spain), 9 Jul. 2000 to 13 Jul. 2000. Spanish Purine Club), which is hereby incorporated by reference in their entirety); or nucleoside transporters.

Useful adenosine transport inhibitors are dipyridamole (Gu, et. al., “Involvement of Bidirectional Adenosine Transporters in the Release of L-[3H]Adenosine from Rat Brain Synaptosomal Preparations,” J Neurochem 64:2105-2110 (1995), which is hereby incorporated by reference in its entirety), nitrobenzylthioinosine, or dilazep (Ki=10⁻¹⁰ to 10⁻⁹M (Baer et al., “Potencies of Mioflazine and Its Derivatives as Inhibitors of Adenosinetransport in Isolated Erythrocytes From Different Species,” J Pharm Pharmacol 42:367-369 (1990), which is hereby incorporated by reference in its entirety)), benzodiazepines (Barker et. al., “Inhibition of Adenosine Accumulation into Guinea Pig Ventricle by Benzodiazepines. Eur J Pharmacol 78:241-244 (1982), which is hereby incorporated by reference in its entirety), dihydropyridies, xanthine, and quinolines derivatives.

Suitable lidoflazine and its analogues include: lidoflazine (Ki=10⁻⁷), mioflazine (Ki=10⁻⁸), soluflazine (Ki=10⁻⁵), 2-(aminocarbonyl)-N-(4-amino-2,6-dichlorophenyl)-4-[5,5-bis(4-fluorophenyl)pentyl]-1-piperazineacetamide (R75231) (Ki=10⁻¹⁰), and draflazine (Ki=10⁻¹⁰).

Suitable benzodiazepines are diazepam (Ki=10⁻⁵-10⁻⁴M), clonazepam (Ki=10⁻⁵-10⁻⁴ M), and midazolam (Ki=10⁻⁶).

Propentofylline is a useful xanthine derivative (Ki=10⁻⁵-10⁻⁴ M) (Parkinson et al., “Effects of Propentofylline on Adenosine A1 and A2 Receptors and Nitrobenzylthioinosine-Sensitive Nucleoside Transporters: Quantitative Autoradiographic Analysis,” Eur J Pharmacol 202:361-366 (1991); Fredholm et al., “Further Evidence That Propentofylline (HWA 285) Influences Both Adenosine Receptors and Adenosine Transport,” Fundam Clin Pharmacol 6:99-111 (1992), which are hereby incorporated by reference in their entirety)).

Suitable quinolinone derivates are cilostazol (IC50=10⁻⁵M (Liu et al., “Inhibition of Adenosine Uptake and Augmentation of Ischemiainduced Increase of Interstitial Adenosine by Cilostazol, An Agent to Treat Intermittent Claudication,” J Cardiovasc Pharmacol 36:351-360 (2000), which is hereby incorporated by reference in its entirety)) and 3-[1-(6,7-diethoxy-2-morpholinoquinazolin-4-yl)piperidin-4-yl]-1,6-dimethyl-2,4(1H,3H)-quinazolinedione hydrochloride (KF 24345) (Ki=10⁻¹⁰-10⁻⁹ M (Hammond et al., “Interaction of the Novel Adenosine Uptake Inhibitor 3-[1-(6,7-Diethoxy-2-Morpholinoquinazolin-4-yl)Piperidin-4-yl]-1,6-Dimethyl-2,4(1H,3H)-Quinazolinedione Hydrochloride (KF24345) With the Es and Ei Subtypes of Equilibrative Nucleoside Transporters,” J Pharmacol Exp Ther 308:1083-1093 (2004), which is hereby incorporated by reference in its entirety)).

Drugs that modulate the concentration of extracellular adenosine and thereby indirectly affect A1 receptor activation.

Extracellular concentration of adenosine can be affected by a series of enzymes that involved in its metabolism. Enzymes involved in adenosine metabolism include: ecto-5′-nucleotidase (CD73) which converts AMP to adenosine; adenosine kinase which catalyzes the process of adenosine to AMP; S-Adenosylhomocysteine hydrolase (SAH-hydrolase) which catalyzes the reversible hydrolysis of S-adenosylhomocysteine (AdoHcy) to adenosine and homocysteine; adenosine uptake or nucleotransport; adenosine diaminase which deaminates the adenosine to inosine. Besides those enzymes that directly affect adenosine metabolism, extracellular concentration of AMP, as a source of extracellular adenosine production, can also effect adenosine concentration. Examples of ecto-5′-nucleotidase modulator according to the present invention, include, without limitation, thiamine monophosphatase (TMPase), Prostatic acid monophosphatase (PAP), and transmenbrane isoform of PAP (TM-PAP).

Suitable inhibitors are S-adenosylhomocysteine hydrolase inhibitors, particularly acyclic adenosine analogues like (Z)-4′,5′-didehydro-5′-deoxy-5′-fluoroadenosine (ZDDFA) (Ki=39.9 nM (Yuan et al., “Mechanism of Inactivation of S-Adenosylhomocysteine Hydrolase by (Z)-4′,5′-Didehydro-5′-Deoxy-5′-Fluoroadenosine,”J Biol Chem 268(23):17030-7 (1993), which is hereby incorporated by reference in its entirety)), methyl 4-(adenine-9-yl)-2-hydroxybutanoate (DZ2002) (Ki=17.9 nM (Wu et al., “Inhibition of S-Adenosyl-L-Homocysteine Hydrolase Induces Immunosuppression,” J Pharmacol Exp Ther 313(2):705-11 (2005), which is hereby incorporated by reference in its entirety)); eritadenine[2(R),3(R)-dihydroxy-4-(9-zdenyl)-butyric acid] (DEA)(Ki=30 nM (Yamada et al., “Structure and Function of Eritadenine and Its 3-Deaza Analogues: Potent Inhibitors of S-Adenosylhomocysteine Hydrolase and Hypocholesterolemic Agents,” Biochem Pharmacol 73(7):981-9 (2007), which is hereby incorporated by reference in its entirety)), 3-deaza-DEA (C3-DEA) (Ki=1.5 μM (Yamada et al., “Structure and Function of Eritadenine and Its 3-Deaza Analogues: Potent Inhibitors of S-Adenosylhomocysteine Hydrolase and Hypocholesterolemic Agents,” Biochem Pharmacol 73(7):981-9 (2007), which is hereby incorporated by reference in its entirety)), and 3-deaza-DEA methylester (C3-OMeDEA) (Ki=1.50 μM (Yamada et al., “Structure and Function of Eritadenine and Its 3-Deaza Analogues: Potent Inhibitors of S-Adenosylhomocysteine Hydrolase and Hypocholesterolemic Agents,” Biochem Pharmacol 73(7):981-9 (2007), which is hereby incorporated by reference in its entirety)).

Useful inhibitors of adenosine deaminase are purine ribosides and 2′-deoxyribosides. The purine ribosides are erythro-9-(2′S-hydroxy-3′R-nonyl)-adenine (EHNA) and its derivatives (Ki=0.51-302 nM (Pragnacharyulu et al., “Adenosine Deaminase Inhibitors: Synthesis and Biological Evaluation of Unsaturated, Aromatic, and Oxo Derivatives of (+)-Erythro-9-(2′S-Hydroxy-3′R-Nonyl)Adenine [(+)-EHNA],” J Med Chem 43(24):4694-700 (2000), which is hereby incorporated by reference in its entirety)). The 2′-deoxyribosides are (2′-deoxycoformycin (pentostatin) and its derivatives (Ki=12-93 μM (Reayi et al., “Inhibition of Adenosine Deaminase by Novel 5:7 Fused Heterocycles Containing the Imidazo[4,5-e][1,2,4]Triazepine Ring System: A Structure-Activity Relationship Study,” J Med Chem 47(4):1044-50 (2004), which is hereby incorporated by reference in its entirety)) as well as acetaminophen (Ki=126 μM at 27° C. (Wang et al., “A Unique Ring-Expanded Acyclic Nucleoside Analogue That Inhibits Both Adenosine Deaminase (ADA) and Guanine Deaminase (GDA; Guanase): Synthesis and Enzyme Inhibition Studies of 4,6-Diamino-8H-1-Hydroxyethoxymethyl-8-Iminoimidazo [4,5-e][1,3]Diazepine,” Bioorg Med Chem Lett 11(22):2893-6 (2001), which is hereby incorporated by reference in its entirety)).

There are mainly two types of inhibitors of adenosine kinase which are similar to adenosine, with one type including the following: 5-iodotubercidin (5-IT) (IC50=26 nM (Ugarkar et al., “Adenosine Kinase Inhibitors. 1. Synthesis, Enzyme Inhibition, and Antiseizure Activity of 5-Iodotubercidin Analogues,” J Med Chem 43(15):2883-93 (2000), which is hereby incorporated by reference in its entirety)), 5-deoxy-5-iodotubercidin (5-d-5-IT), (IC 50=0.9 nm) (Ugarkar et al., “Adenosine Kinase Inhibitors. 1. Synthesis, Enzyme Inhibition, and Antiseizure Activity of 5-Iodotubercidin Analogues,” J Med Chem 43(15):2883-93 (2000), which is hereby incorporated by reference in its entirety) and IC50=1.09 nM (Muchmore et al., “Crystal Structures of

Human Adenosine Kinase Inhibitor Complexes Reveal Two Distinct Binding Modes,” J Med Chem 49(23):6726-31 (2006), which is hereby incorporated by reference in its entirety)), and 5-amino-5′-deoxy analogues of 5-bromo-and 5-iodotubercidine. The other type of inhibitor of adenine kinase is a non-nucleoside like, such as alkynylpyrimidine class (5-(4-dimethylamino)phenyl)-6-(6-morpholin-4-ylpyrodin-3-ylethynyl)pyrimidin-4-ylamne (IC 50=68 nM) (Muchmore et al., “Crystal Structures of Human Adenosine Kinase Inhibitor Complexes Reveal Two Distinct Binding Modes,” J Med Chem 49(23):6726-31 (2006), which is hereby incorporated by reference in its entirety), Gp-1-515 (IC 50=206 nM (Firestein et al., “Inhibition of Neutrophil Adhesion by Adenosine and an Adenosine Kinase Inhibitor. The Role of Selectins,” J Immunol 154(1):326-34 (1995), which is hereby incorporated by reference in its entirety)), 4-(N-phenylamino)-5-phenyl-7-(59-deoxyribofuranosyl)pyrrolo[2,3-c]pyrimidine (GP683), N7-((1′R,2′S,3′R,4′S)-2′,3′-dihydroxy-4′-amino-cyclopentyl)-4-amino-5-bromo-pyrrolo[2,3-a]pyrimidine (A-286501) (IC 50=0.47 nM (Jarvis et al., “Analgesic and Anti-Inflammatory Effects of A-286501, a Novel Orally Active Adenosine Kinase Inhibitor,” Pain 96(1-2):107-18 (2002), which is hereby incorporated by reference in its entirety)), 4-amino-5-(3-bromophenyl)-7-(6-morpholino-pyridin-3-yl)pyrido[2,3-d]pyrimidine (ABT702) (IC 50=1.7 nM (Jarvis et al., “ABT-702 (4-amino-5-(3-bromophenyl)-7-(6-morpholinopyridin-3-yl)pyrido[2,3-d]pyrimidine), A Novel Orally Effective Adenosine Kinase Inhibitor With Analgesic and Anti-Inflammatory Properties: I. In Vitro Characterization and Acute Antinociceptive Effects In The Mouse,” J Pharmacol Exp Ther 295(3):1156-64 (2000), which is hereby incorporated by reference in its entirety)), A-134974 (IC 50=60 μM (McGaraughty et al., “Effects of A-134974, a Novel Adenosine Kinase Inhibitor, on Carrageenan-Induced Inflammatory Hyperalgesia and Locomotor Activity in Rats: Evaluation of the Sites of Action,” J Pharmacol Exp Ther 296(2):501-9 (2001), which is hereby incorporated by reference in its entirety)), and many other derivatives. The two class of inhibitors bind two significantly different protein conformational states of their target structure.

Adenosine modulators are divided into the following types: Ecto-NTPDase inhibitors, ATP analogues that are non-hydrolysable P2 receptor agonists, P2 receptor antagonists, and non-ATP analogues.

Ecto-5′-nucleotidase CD73 modulators includes inhibitors of the enzyme and activators of the enzyme. Examples of inhibitors of the enzyme are sodium nitroprussside (SNP), foskolin, and giberclamide (IC50=10.5 μM (Sato et al., “The Effect of Glibenclamide on the Production of Interstitial Adenosine by Inhibiting Ecto-5-Nuceotidase in Rat Hearts,” Br J Pharm 122:611-618 (1997), which is hereby incorporated by reference in its entirety)). Tyramin is a suitable activator of the enzyme.

Suitable ATP analogues that are non-hydrolysable P2 receptor antagonists are 8-Bus-ATP (Ki=10 μM (Gendron et al., “Novel Inhibitors of Nucleoside Triphosphate Diphosphohydrolases: Chemical Synthesis and Biochemical and Pharmacological Characterizations,” J Med Chem 43(11):2239-47 (2000), which is hereby incorporated by reference in its entirety)), 8-hexylS-ATP (Ki=16 μM (Gendron et al., “Novel Inhibitors of Nucleoside Triphosphate Diphosphohydrolases: Chemical Synthesis and Biochemical and Pharmacological Characterizations,” J Med Chem 43(11):2239-47 (2000), which is hereby incorporated by reference in its entirety)), 8-CH2BuS-ATP (Ki=45 μM (Gendron et al., “Novel Inhibitors of Nucleoside Triphosphate Diphosphohydrolases: Chemical Synthesis and Biochemical and Pharmacological Characterizations,” J Med Chem 43(11):2239-47 (2000), which is hereby incorporated by reference in its entirety)), ATPγS (pIC 50=5.2 (Chen et al., “Inhibition of Ecto-ATPase by the P2 Purinoceptor Agonists, ATPgammaS, Alpha,Beta-Methylene-ATP, and AMP-PNP, in Endothelial Cells,” Biochem Biophys Res Commun 233:442-446 (1997), which is hereby incorporated by reference in its entirety)), AMP-PNP (pIC 50=4.0 (Chen et al., “Inhibition of Ecto-ATPase by the P2 Purinoceptor Agonists, ATPgammaS, Alpha,Beta-Methylene-ATP, and AMP-PNP, in Endothelial Cells,” Biochem Biophys Res Commun 233:442-446 (1997), which is hereby incorporated by reference in its entirety)), and α,β-MeATP (pIC 50=4.5 (Chen et al., “Inhibition of Ecto-ATPase by the P2 Purinoceptor Agonists, ATPgammaS, Alpha,Beta-Methylene-ATP, and AMP-PNP, in Endothelial Cells,” Biochem Biophys Res Commun 233:442-446 (1997), which is hereby incorporated by reference in its entirety)).

Useful P2 receptor antagonists are suramin (Ki=44 μM (Chen et al., “Inhibition of Ecto-ATPase by the P2 Purinoceptor Agonists, ATPgammaS, Alpha,Beta-Methylene-ATP, and AMP-PNP, in Endothelial Cells,” Biochem Biophys Res Commun 233:442-446 (1997), which is hereby incorporated by reference in its entirety)), (pIC 50=4.57 (Yegutkin et al., “Inhibitory Effects of Some Purinergic Agents on Ecto-ATPase Activity and Pattern of Stepwise ATP Hydrolysis in Rat Liver Plasma Membranes,” Biochim Biophys Acta 1466(1-2):234-44 (2000), which is hereby incorporated by reference in its entirety)), and (IC 50=4604-114 μM (Crack et al., “Pharmacological and Biochemical Analysis of FPL 67156, a Novel, Selective Inhibitor of Ecto-ATPase,” Br J Pharmacol 114(2):475-81 (1995); Dowd et al., “Inhibition of Rat Parotid Ecto-ATPase Activity,” Arch Oral Biol 44(12):1055-1062 (1999); Stout et al., “Inhibition of Purified Chicken Gizzard Smooth Muscle Ecto-ATPase by P2 Purinoceptor Antagonists,” Biochem Mol Biol Int 36:927-934 (1995), which are hereby incorporated by reference in their entirety)), reactive blue (pIC 50=4.3 (Yegutkin et al., “Inhibitory Effects of Some Purinergic Agents on Ecto-ATPase Activity and Pattern of Stepwise ATP Hydrolysis in Rat Liver Plasma Membranes,” Biochim Biophys Acta 1466(1-2):234-44 (2000), which is hereby incorporated by reference in its entirety)) and (IC 50=2804 (Dowd et al., “Inhibition of Rat Parotid Ecto-ATPase Activity,” Arch Oral Biol 44(12):1055-1062 (1999), which is hereby incorporated by reference in its entirety)), Coomassie brilliant blue R (IC 50=114 μM (Dowd et al., “Inhibition of Rat Parotid Ecto-ATPase Activity,” Arch Oral Biol 44(12):1055-1062 (1999), which is hereby incorporated by reference in its entirety)), 4,4′diisothiocyanatostilbene-2,2′disulphonec acid (DIDS) (IC 50=150 μM (Dowd et al., “Inhibition of Rat Parotid Ecto-ATPase Activity,” Arch Oral Biol 44(12):1055-1062 (1999), which is hereby incorporated by reference in its entirety)), and 4-acetamido-4′-isothiocyanatostilbene-2,3-′-disulphonic acid (SITS) (IC 50=500 μM (Drakulich et al., “Effect of the Ecto-ATPase Inhibitor, ARL67156, on the Bovine Chromaffin Cell Response to ATP,” Eur J Pharmacol 485(1-3):137-40 (2004), which is hereby incorporated by reference in its entirety)).

Non-ATP analogues, without or with only weak effect on purinoceptors include: ARL67156 (FPL67156) (Ki=0.255 μM (Drakulich et al., “Effect of the Ecto-ATPase Inhibitor, ARL67156, on the Bovine Chromaffin Cell Response to ATP,” Eur J Pharmacol 485(1-3):137-40 (2004), which is hereby incorporated by reference in its entirety)), (IC50=4.6 μM (Crack et al., “Pharmacological and Biochemical Analysis of FPL 67156, a Novel, Selective Inhibitor of Ecto-ATPase,” Br J Pharmacol 114(2):475-81 (1995), which is hereby incorporated by reference in its entirety)), and (IC50=120 μM (Dowd et al., “Inhibition of Rat Parotid Ecto-ATPase Activity,” Arch Oral Biol 44(12):1055-1062 (1999), which is hereby incorporated by reference in its entirety)). This is a selective inhibitor of ecto-ATPase and has a lack of or only has weak effect on P2 receptors.

Agents of the present invention can be administered orally, parenterally, for example, subcutaneously, intravenously, intramuscularly, intraperitoneally, by intranasal instillation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The active agents of the present invention may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active agents may be incorporated with excipients and used in the form of tablets, capsules, elixirs, suspensions, syrups, and the like. Such compositions and preparations should contain at least 0.1% of active agent. The percentage of the agent in these compositions may, of course, be varied and may conveniently be between about 2% to about 60% of the weight of the unit. The amount of active agent in such therapeutically useful compositions is such that a suitable dosage will be obtained. Preferred compositions according to the present invention are prepared so that an oral dosage unit contains between about 1 and 250 mg of active agent.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to the active ingredient, sucrose as a sweetening agent, methyl and propylparabens as preservatives, a dye, and flavoring such as cherry or orange flavor.

These active agents may also be administered parenterally. Solutions or suspensions of these active agents can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The agents of the present invention may also be administered directly to the airways in the form of an aerosol. For use as aerosols, the agents of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

In another embodiment of the present invention, the step of administering can be carried out with Tecadensor, CVT-3619, BAY-68-4986, INFO-8875, and/or BTJ-009.

There are many theories to explain the physiological functions of acupuncture/acupressure in the basic mechanism of pain. One is the “Chinese Meridian” (pathway) theory where perhaps acupressure stimulates nerve endings with the release of pain killing endorphins Another is the “Gate Control Theory” where sensor stimulation (acupressure) sends pleasurable impulses to the brain at a rate four times faster than painful stimuli. These impulses shut the neural “GATES” so that the slower messages of pain are blocked from reaching the brain. This “Counter Stimulation” overloads the neurons in the spinal cord, thereby preventing the perception of pain.

It has been found that stimulation of a site on the body proper (i.e., ear, hand), converts a message into a nerve impulse that is transmitted to the brain. This “counterstimulation” message finally reaches the pituitary gland and promotes it to release enkephalins and endorphins These neural opiate-like pain killing peptides block the perception of pain. Widespread clinical material dating from ancient times testifies to the effectiveness of kneading or pressing certain points on the body in stopping pain.

Acupuncture is useful in treating a number of disorders according to their degree of responsiveness. Acupuncture/Acupressure is considered to be very effective in treating headaches. Muscle contractures, no matter how chronic, are most always quickly relieved. Statistics indicate success in 90% of cases involving pain treated by acupuncture/acupressure.

### EXAMPLES

The following examples are provided to illustrate embodiments of the present invention but are by no means intended to limit its scope.

### Example 1

### Surgery, Experimental Models, Behavioral Assessment, CCPA Administration, and Acupuncture

The Institutional Animal Care and Use Committee at University of Rochester approved all procedures in this study. The minimum number of animals needed to achieve statistical significance was used as per direction of the International Society for the Study of Pain Guidelines (Covino et al., “Ethical Standards for Investigations of Experimental Pain in Animals,” Pain 90 (1980), which is hereby incorporated by reference in its entirety).

C57BL/6J mice (8-10 weeks of age) were used in all experiments. Al receptor knock out mice ref and A2a receptor knockout mice ref were on C57BL/6 genetic background and WT littermate used as controls. All studies were carried out in a quiet room to which the mice were habituated for at least 1-2 weeks.

Peripheral inflammation was induced by injection of Complete Freud Adjuvant (CFA, mixed with an equal amount of oil, total volume 0.1 ml) in the plantar surface of the left hind paw of mice (25-30 g, Jackson labs) (Raghavendra et al., “Complete Freunds Adjuvant-Induced Peripheral Inflammation Evokes Glial Activation and Proinflammatory Cytokine Expression in the CNS,” Eur. J. Neurosci. 20:467-473 (2004), which is hereby incorporated by reference in its entirety). An equal amount of saline (0.1 ml) was injected in the right hind paw as control. Neuropathic pain was induced by ligation of the sciatic nerve with 4.0 polypropylene suture in mice sedated with ketamine (60 mg/kg, i.p.) and xylazine (10 mg/kg, i.p.) (Bennett et al., “A Peripheral Mononeuropathy in Rat that Produces Disorders of Pain Sensation Like Those Seen in Man,” Pain 33:87-107 (1988); Martucci et al., “The Purinergic Antagonist PPADS Reduces Pain Related Behaviours and Interleukin-1 beta, Interleukin-6, iNOS and nNOS Overproduction in Central and Peripheral Nervous System After Peripheral Neuropathy in Mice,” Pain 137:81-95 (2008), which are hereby incorporated by reference in their entirety).

Mechanical allodynia was evaluated using repeated stimulations with a Von Frey filament exerting 0.02 grams of force onto the plantar surface (Colburn et al., “The Effect of Site and Type of Nerve Injury on Spinal Glial Activation and Neuropathic Pain Behavior,” Exp. Neurol. 157:289-304. (1999), which is hereby incorporated by reference in its entirety). The percentage of negative responses of a total of 10 trials was calculated for each foot. Thermal hyperalgesia was assessed using an Analgesymeter (Ugo Basile, Comerio, Italy) (Stein et al., “Intrinsic Mechanisms of Antinociception in Inflammation: Local Opioid Receptors and Beta-Endorphin,” J. Neurosci. 10:1292-1298 (1990), which is hereby incorporated by reference in its entirety). In short, a mobile radiant heat source was focused on the hind paw, and the paw withdrawal latencies were defined as the time taken by the mouse to remove its hind paw from the heat source (max 20 sec to avoid tissue damage). The paw withdrawal was repeated three times for each foot and the average calculated. To avoid conditioning to stimulation a 5 min rest period was interposed between each trial in both thermal and mechanical tests. Behavioral parameters were evaluated prior to intraplantar injection of CFA or nerve ligation (i.e. day 0), and again on day 3-4 in mice receiving the CFA injection, and at day 5-7 in mice with nerve ligation unless otherwise noted. Prior to injection of CCPA, saline, or acupuncture in the Zusanli point, the mice were placed in a restraining under light isoflurane anesthesia (˜1%). The total duration of anesthesia was ˜2 min and mice with inflammatory, neurogenic pain, and their controls were treated similarly. 2-chloro-N6-cyclopentyl-adenosine (CCPA, 0.1 mM, 20 μl) was injected in the Zusanli point ˜5 min before acupuncture. For acupuncture, a small acupuncture needle, 0.16×13 mm (08-02, Lhass Medical Inc, Accord, Mass.) was gently inserted in a depth of 1.5 mm in the Zusanli point (ST36) located 3-4 mm below and lateral 1-2 mm for the midline of the knee (Kim et al., “Analgesic Effects by Electroacupuncture Were Decreased in Inducible Nitric Oxide Synthase Knockout Mice,” Neural. Res. 29(Suppl. 1):S28-31 (2007); Roh et al., “Bee Venom Injection Significantly Reduces Nociceptive Behavior in the Mouse Formalin Test Via Capsaicin-Insensitive Afferents,” J. Pain 7:500-512 (2006), which are hereby incorporated by reference in their entirety). The needle was slowly rotated ˜2 time (˜20-30 sec) every 5 min for a total of 30 min during an acupuncture session. A microdialysis probe (MD-2211, Bioanalytical systems, West Lafayette, Ind.) was implanted 1-3 hrs prior to collection of microdialysis samples. The microdialysis probe was implanted in a distance of 0.4-0.6 mm from the Zusanli point. The microdialysis probe was perfused with Ringer's solution at a rate of 1 μl per minute. The microdialysates were collected on ice and the perfusate collected over a 30 min period (30 μl) was immediately frozen at −80° C. until HPLC analysis. Deoxycoformycin was administered in a dose of 50 mg/kg i.p. 30 min prior to acupuncture.

### Example 2

### In vivo Electrophysiology

Mice were anaesthetized with 2-3% isoflurane, intubated, and artificially ventilated with a small animal ventilator (SAAR-830, CWE). Body temperature was monitored by a rectal probe and maintained at 37° C. by a heating blanket (BS4, Harvard Apparatus). A craniotomy (1-1.5 mm in diameter), centered 0.1 mm anterior to the bregma and 1.5 mm lateral from midline, was made over the left anterior cingulated cortex. A custom-made metal plate was glued to the skull with dental acrylic cement. The mice were for the remaining part of the experiment maintained at 2% isoflurane. LFP recordings were obtained from layer 4 of anterior cingulate cortex (ACC), 0.8 mm below the pial surface by a patch pipette (TW100E-4, WPI; outer diameter, 1.0 mm; inner diameter, 0.75 mm; tip diameter, 1-2 μm). LFP signals were amplified, bandpass filtered at (1-100 Hz) and digitized at 10 kHz as previously described (Bekar et al., “Adenosine is Crucial for Deep Brain Stimulation-Mediated Attenuation of Tremor,” Nat. Med. 14:75-80 (2008); Wang et al., “Astrocytic Ca2+ Signaling Evoked by Sensory Stimulation In vivo,” Nat. Neurosci. 9:816-823 (2006), which is hereby incorporated by reference in its entirety). Dura matter was kept intact. A custom-made bipolar electrode was inserted subcutaneously into the right hindpaw. High intensity stimulation (10 mA, 20 ms) were evoked every 120 sec. Lower stimulation intensities evoked either no or variable responses consistent with the idea that that ACC neurons respond primarily to painful stimuli Wei et al., “Potentiation of Sensory Responses in the Anterior Cingulate Cortex Following Digit Amputation in the Anaesthetised Rat,” J. Physiol. 532:823-833 (2001), which is hereby incorporated by reference in its entirety). The amplitude of the field EPSPs was measured using the pCLAMP 9.2 program (Axon Instruments, Inc., Foster City, Calif., USA).

### Example 3

### HPLC Analysis of Purines

The analysis of enzymatic degradation of purines was based on sections (400 μm) of skeletal muscles with overlying subcutis harvested from tissue in the Zusanli point. Each section per well was placed into a 6-well plate with 2 ml (in a phosphate-free buffer (in mM: 2 CaCl₂, 120 NaCl, 5 KCL, 10 Glucose, 20 HEPES, pH=7.3) and bubble with 100% O₂ containing 1 mM AMP with or without 500 μM deoxycoformycin (Tocris Bioscience, UK). The samples were collected from each well at 0 and 45 min and stored at −80° C. for HPLC analysis. The analyses were carried out on an ESA reverse-phase H584 HPLC system (ESA Inc., USA) and an ESA model 526 UV detector (ESA Inc.) as previously described (Cui et al., “The Organic Cation Transporter-3 is a Pivotal Modulator of Neurodegeneration in the Nigrostriatal Dopaminergic Pathway,” Proc. Nat'l. Acad. Sci. USA 106:8043-8048 (2009); Volonte et al., “Development of an HPLC Method for Determination of Metabolic Compounds in Myocardial Tissue,” J. Pharm. Biomed. Anal. 35:647-653 (2004), which are hereby incorporated by reference in their entirety). Chromatographic separation was achieved by using a Lichrospher® 100 RP-18 column (5 μm, 250 mm×3 mm; Merck, Germany). The mobile phase consisted of 215 mM KH₂PO₄, 2.3 mM tetrabutylammonium bisulfate (TBAHS), 3.2% (v/v) acetonitrile (HPLC grade) and HPLC grade water, pH 6.2. The flow rate was maintained at 0.4 ml/min. Daily calibration curves were prepared by a four point standard (3, 1, 0.3 or 0.1 uM) of ATP, ADP, AMP, adenosine, inosine and IMP in 0.4 M perchloric acid, respectively. Eluted purines were detected at 260 nm, and the chromatographic peaks were integrated using CoulArray software. Pharmacological analysis of enzymes involved in extracellular degradation of AMP was measured using the Malachite Green Phosphate Detection Kit (Fisher et al., “A Sensitive, High-Volume, Colorimetric Assay for Protein Phosphatases,” Pharm. Res. 11:759-763 (1994), which is hereby incorporated by reference in its entirety) in samples collected from sections were incubated in AMP (1 mM) in a phosphate-free Ringer solution.

### Example 4

### Effect of Acupuncture on Extracellular Concentration of Adenosine

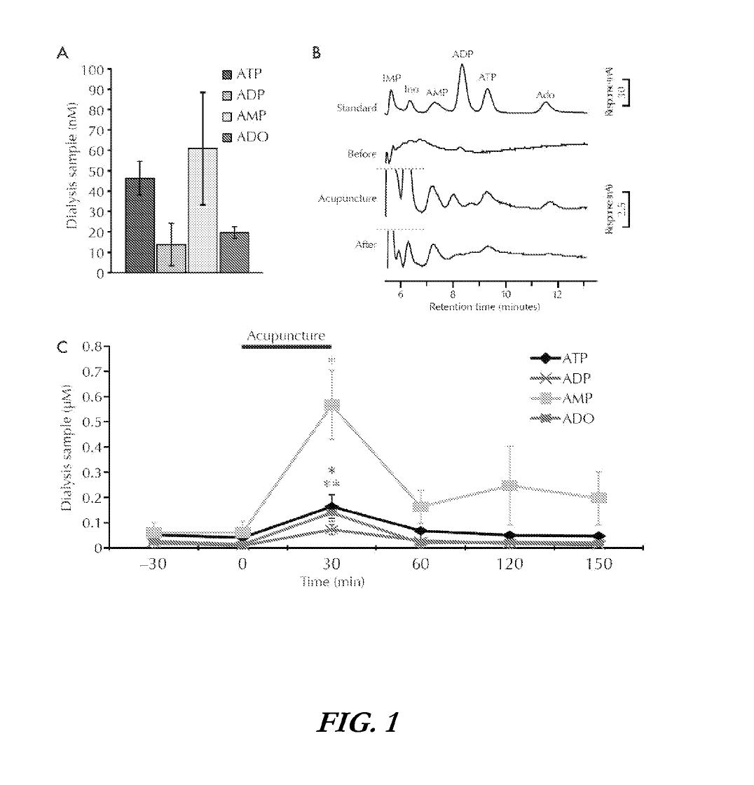

Adenosine is a breakdown product of the energy metabolite ATP, which is released in response to both mechanical and electrical stimulation, or heat (Bekar et al., “Adenosine is Crucial for Deep Brain Stimulation-Mediated Attenuation of Tremor,” Nat. Med. 14:75-80 (2008); Davalos et al., “ATP Mediates Rapid Microglial Response to Local Brain Injury In vivo,” Nat. Neurosci. (2005); Schachter, S. C., “Complementary and Alternative Medical Therapies,” Curr. Opin. Neurol. 21:184-189 (2008); Wang et al., “P2X7 Receptor Inhibition Improves Recovery After Spinal Cord Injury,” Nat. Med. 10:821-827 (2004), which are hereby incorporated by reference in their entirety). Adenosine is also an analgesic agent that suppresses pain through Gi-coupled A1-adenosine receptors (Maione et al., “The Antinociceptive Effect of 2-chloro-2′-C-methyl-N6-Cyclopentyladenosine (2′-Me-CCPA), a Highly Selective Adenosine A1 Receptor Agonist, in the Rat,” Pain 131:281-292 (2007); Poon et al., “Antinociception by Adenosine Analogs and Inhibitors of Adenosine Metabolism in an Inflammatory Thermal Hyperalgesia Model in the Rat,” Pain 74:235-245 (1998); Sjolund et al., “Adenosine Reduces Secondary Hyperalgesia in Two Human Models of Cutaneous Inflammatory Pain,” Anesth. Analg. 88:605-610 (1999); Zahn et al., “Adenosine A1 but not A2a Receptor Agonist Reduces Hyperalgesia Caused by a Surgical Incision in Rats: A Pertussis Toxin-Sensitive G Protein-Dependent Process,” Anesthesiology 107:797-806 (2007), which are hereby incorporated by reference in their entirety). To determine whether adenosine play a role the analgesic effects of acupuncture, it was initially asked whether the extracellular concentration of adenosine increases during acupuncture. Samples of the interstitial fluid were collected by a microdialysis probe implanted in the tibialis anterior muscle/subcutis in a distance of 0.4-0.6 mm from the “Zusanli point”. Adenine nucleotides, adenosine, and inosine were quantified using high-performance liquid chromatography (HPLC) with UV absorbance before, during and after acupuncture (Volonte et al., “Development of an HPLC Method for Determination of Metabolic Compounds in Myocardial Tissue,” J. Pharm. Biomed. Anal. 35:647-653 (2004), which is hereby incorporated by reference in its entirety). During baseline condition, the concentration of ATP, ADP, AMP, and adenosine were in the low nM range (FIG. 1A) in accordance with previous reports (Li et al., “ATP Concentrations and Muscle Tension Increase Linearly with Muscle Contraction,” J. Appl. Physiol. 95:577-583 (2003); Li et al., “Interstitial ATP and Norepinephrine Concentrations in Active Muscle,” Circulation 111:2748-2751 (2005), which are hereby incorporated by reference in their entirety). Acupuncture applied by gentle manual rotation of the acupuncture needle every 5 min for a total of 30 min sharply increased the extracellular concentration of all purines detected (FIG. 1B). Adenosine increased ˜7-fold (139.2±34.1 from 19.7±2.9 nM) during the 30 min acupuncture session. The extracellular concentration of purines returned to baseline after acupuncture, with the exception of AMP, which remained elevated for the duration of the experiment (FIG. 1C). It is in this regard of interest that previous studies have shown that deep brain stimulation (DBS) also is linked to a sharp increase in the extracellular accumulation of ATP and adenosine. Similar to electroacupuncture or transcutaneous electrical nerve stimulation, deep brain stimulation delivers electrical stimulation, and the extracellular concentration of adenosine increase several-fold during the stimulation (Bekar et al., “Adenosine is Crucial for Deep Brain Stimulation-Mediated Attenuation of Tremor,” Nat. Med. 14:75-80 (2008), which is hereby incorporated by reference in its entirety).

### Example 5

### Impact of Adenosine on Analgesic Effects of Acupuncture

Having established that adenosine is released during acupuncture, the next question asked was whether adenosine mediates the analgesic effects of acupuncture. At a first level of analysis, the analgesic effect of the selective A1 receptor agonist, 2-chloro-N(6)-cyclopentyladenosine (CCPA) (Lohse et al., “2-Chloro-N6-Cyclopentyladenosine: A Highly Selective Agonist at Al Adenosine Receptors,” Naunyn Schmiedebergs Arch. Pharmacol. 337:687-689 (1988), which is hereby incorporated by reference in its entirety) was tested in two mice models of chronic pain. In the first set of experiments, inflammatory pain was evoked by injection of complete Freund's adjuvant (CFA) in the right paw (Raghavendra et al., “Complete Freunds Adjuvant-Induced Peripheral Inflammation Evokes Glial Activation and Proinflammatory Cytokine Expression in the CNS,” Eur. J. Neurosci. 20:467-473 (2004), which is hereby incorporated by reference in its entirety) (FIG. 2A). The mice developed following injection of CFA mechanical allodynia to innocuous stimulation with Von Frey filaments of the ipsilateral paw peaking at day 4 to 5, as well as thermal allodynia detected as a significant decrease in withdrawal latency to heat (Abdi et al., “The Effects of KRN5500, a Spicamycin Derivative, on Neuropathic and Nociceptive Pain Models in Rats,” Anesth. Analg. 91:955-959 (2000), which is hereby incorporated by reference in its entirety). Administration of CCPA in the right Zusanli point (ST36) (Kim et al., “Analgesic Effects by Electroacupuncture Were Decreased in Inducible Nitric Oxide Synthase Knockout Mice,” Neurol. Res. 29(Suppl. 1):528-31 (2007); Roh et al., “Bee Venom Injection Significantly Reduces Nociceptive Behavior in the Mouse Formalin Test Via Capsaicin-Insensitive Afferents,” J. Pain 7:500-512 (2006), which are hereby incorporated by reference in their entirety) evoked a sharp increase in the threshold to touch (FIG. 2B) and thermal pain (FIG. 2C). Mechanical stimulation was better tolerated and touch sensitivity increased from 38.0±2.5 to 85.7±2.0%; p<0.01, Tukey-Kramer, whereas thermal sensitivity was almost gone (paw withdrawal increased from 3.7±0.3 to 12.7±1.5 sec; p<0.05 following administration of CCPA. Similarly, mechanical allodynia was sharply reduced by CCPA in mice suffering from neuropathic pain (touch sensitivity improved from 25.0±2.2 to 83.3±4.9%; p<0.01, concurrently with a reduction of the sensitivity to thermal pain (withdrawal of foot increased from 2.9±0.3 to 16.2±2.4 sec; p<0.01). Injection of CCPA in the left leg did not alter the pain threshold in the right leg indicating that the action of CCPA was mediated by activation of local A1 receptors (FIG. 5). To exclude that pathways other than A1 receptors were implicated in the anti-analgesic effect of CCPA, the effect of CCPA in mice was compared with deletion of the adenosine receptor A1 with wildtype littermates (Sun et al., “Mediation of Tubuloglomerular Feedback by Adenosine: Evidence from Mice Lacking Adenosine 1 Receptors,” Proc. Natl. Acad. Sci. USA 98:9983-9988 (2001), which is hereby incorporated by reference in its entirety). This strategy provided direct evidence for the necessity of A1 receptor expression in CCPA-mediated pain suppression: While CCPA effectively reduced mechanical and thermal pain in wildtype mice suffering from inflammatory pain, CCPA had no clinical benefit in mice with deletion of A1 receptors (FIG. 2B-C). Thus, the anti-nociceptive and anti-hyperalgesic effects of CCPA required adenosine A1 receptors expression.

Neuropathic pain was next modeled by spared injury of the sciatic nerve (Vadakkan et al., “A Behavioral Model of Neuropathic Pain Induced by Ligation of the Common Peroneal Nerve in Mice,” J. Pain 6:747-756 (2005), which is hereby incorporated by reference in its entirety), in which pain peaked 5-7 days after nerve ligation (FIG. 2D). CCPA injected in the Zusanli point reduced neuropathic with an efficacy that compared to its suppression of inflammatory pain (FIG. 2E-F). The pain-relieving effect of CCPA was in both pain models transient, and did not alter sensitivity to painful stimulation in the contralateral (left) leg. Also, injection of CCPA in the left leg did not alter the pain threshold in the right leg (FIG. 5). Substituting the CCPA injection with an equal volume of saline (control vehicle) failed to change the threshold to either thermal or mechanical induced pain.

To understand how CCPA reduced the sensitivity to painful stimulation, and specifically address whether CCPA acted directly on ascending nerve tracks, in vivo responses to painful stimulation foot shock of the right foot in the left anterior cingulate cortex were recorded (ACC) (FIG. 2G). The ACC has experimentally been shown to play a pivotal role in perception of pain (Wei et al., “Potentiation of Sensory Responses in the Anterior Cingulate Cortex Following Digit Amputation in the Anaesthetised Rat,” J. Physiol. 532:823-833 (2001), which is hereby incorporated by reference in its entirety) and painful electrical nerve stimulation is in human linked to activation of the anterior cingulated cortex (Davis et al., “Functional MRI of Pain- and Attention-Related Activations in the Human Cingulate Cortex,” J. Neurophysiol. 77:3370-3380 (1997), which is hereby incorporated by reference in its entirety). High intensity stimulation (10 mA, 20 ms) evoked consistent field excitatory postsynaptic potentials (fEPSP) in the ACC with a latency of ˜40 msec, reflecting the involvement of a polysynaptic pathway, including primary afferents, as well as spinothalamic and thalamocortical tracts. Lower stimulation intensities evoked either no or variable responses consistent with the idea that that ACC neurons respond primarily to painful stimuli (Devinsky et al., “Contributions of

Anterior Cingulate Cortex to Behaviour,” Brain 118(Pt 1):279-306 (1995), which is hereby incorporated by reference in its entirety). After recording the responses to foot shock during baseline conditions for a total 20 min, CCPA (0.1 mM. 20 μl) was injected in the Zusanli point in the left leg or contralateral to the left foot receiving the painful stimuli. CCPA administered contralateral to the painful stimulation had no effect on fEPSP excluding the possibility that CCPA acted centrally (FIG. 2G). In contrast, CCPA injected in the Zusanli point in the right leg or ipsilateral to the painful stimulation induced a striking decrease in the amplitude at the fEPSP amplitude. The decline of the fEPSP amplitude occurred as early as 6 min after the CCPA injection and the fEPSP amplitude felt from an average of ˜0.65 mV before injection to ˜0.22 mV within 20 min, which represent a drop to 26.6±11.0% of baseline values. Combined, this analysis suggests that CCPA acts locally, likely on unmyelinated C-fibers in the superficial peroneal nerve, which travels in close proximity to the Zusanli points. It is unlikely that CCPA within six minutes can diffuse over a distance of 1.8-2.0 mm and bind to Al receptors on the afferent nerve terminal in the foot (Karlsten et al., “Local Antinociceptive and Hyperalgesic Effects in the Formalin Test After Peripheral Administration of Adenosine Analogues in Mice,” Pharmacol. Toxicol. 70:434-438 (1992); Sawynok et al., “Peripheral Antinociceptive Effect of an Adenosine Kinase Inhibitor, With Augmentation by an Adenosine Deaminase Inhibitor, in the Rat Formalin Test,” Pain 74:75-81 (1998), which are hereby incorporated by reference in their entirety). Similarly, the presynaptic terminals of dorsal root ganglion cells located in the substantia gelatinosa (SG) of the spinal cord is potently inhibited by A1 receptor agonists (Lao et al., “Modulation by Adenosine of Adelta and C primary-Afferent Glutamatergic Transmission in Adult Rat Substantia Gelatinosa Neurons,” Neuroscience 125:221-231 (2004b); Schulte et al., “Distribution of Antinociceptive Adenosine A1 Receptors in the Spinal Cord Dorsal Horn, and Relationship to Primary Afferents and Neuronal Subpopulations,” Neuroscience 121:907-916 (2003); Nakamura et al., “Characterization of Adenosine Receptors Mediating Spinal Sensory Transmission Related to Nociceptive Information in the Rat,” Anesthesiology 87:577-584 (1997); Reeve et al., “Electrophysiological Study on Spinal Antinociceptive Interactions Between Adenosine and Morphine in the Dorsal Horn of the Rat,” Neurosci. Lett. 194:81-84 (1995), which are hereby incorporated by reference in their entirety). The distance of the terminals from the Zusanli point (˜3.0-3.2 mm) makes it, however, unlikely that CCPA within six minutes can diffuse over such a great distance and inhibit synaptic transmission in substantia gelatinosa. Mice with deletion of A1 receptors failed to respond to CCPA injection in sharp contrast to the potent depression of the amplitude of fEPSP in WT mice (FIG. 2G). Combined, this set of studies indicated that CCPA reduced painful stimulation by activating adenosine A1 receptors on unmyelinated C-fibers and possible As-fibers in the superficial peroneal nerve.

### Example 6

### Effect of Acupuncture on Inflammatory and Neuropathic Pain

Does adenosine released during acupuncture mediate the anti-nociceptive and anti-hyperalgesic effects of acupuncture? To address this issue, the effects of acupuncture on inflammatory and neuropathic pain were next evaluated. A needle was gently inserted 1.5 mm deep in the Zusanli point and rotated once every 5 min for 30 min to mimic a typical acupuncture session (FIG. 3A). Animals suffering from either inflammatory or neuropathic pain clearly benefited from the acupuncture treatment: Touch sensitivity fell from 27.5±2.3 to 67.1±4.0%; p<0.01, Tukey-Kramer, whereas the threshold to thermal pain increased from 3.9±0.4 to 10.6±0.8 sec; p<0.01, in animals with inflammatory pain (FIG. 3B-C). Similarly, mechanical allodynia was sharply reduced by acupuncture in mice suffering from neuropathic pain. Touch sensitivity improved from 26.7±4.9 to 71.7±4.8%; p<0.01, Tukey-Kramer, concurrently with a reduction of the sensitivity to thermal pain (withdrawal of foot increased from 3.3±0.3 to 9.4±0.9 sec; p<0.01, Tukey-Kramer, FIG. 3D-F). Similar to CCPA injection (FIG. 2), and consistent with earlier publications (Kim et al., “Analgesic Effects by Electroacupuncture Were Decreased in Inducible Nitric Oxide Synthase Knockout Mice,” Neurol. Res. 29(Suppl. 1):528-31 (2007); Zhao, Z. Q., “Neural Mechanism Underlying Acupuncture Analgesia,” Prog. Neurobiol. 85:355-375 (2008), which are hereby incorporated by reference in their entirety), acupuncture-mediated pain suppression was transient and the hypersensitivity to both tactile and thermal stimulation had returned to pre-acupuncture levels the following day. Most significantly, acupuncture failed to reduce pain in A1 KO mice. The hypersensitivity to either mechanical or thermal pain persisted in mice with deletion of adenosine A1 receptors in contrast to their littermate wildtype controls, which clearly benefitted from acupuncture (FIG. 3B-C, E-F).

### Example 7

### Effect on Painful Stimulation of Adenosine Released During Acupuncture

A remaining question is whether adenosine released during acupuncture, similar to CCPA, reduced input to the ACC in response to painful stimulation. Using a similar strategy as in FIG. 2G, the effect of acupuncture on the amplitude of fEPSP recorded in the left ACC evoked by painful foot shock in the right leg were assessed (FIG. 3G). Similar to the CCPA injection, acupuncture in the left Zusanli point had no significant effect on the fEPSP in response to painful stimulation. However, acupuncture in the right Zusanli point (ipsilateral to stimulation) suppressed eEPSP and the inhibition continued to increase in potency during the observation period. The amplitude of fEPSP was maximally reduced to 53.7±7.2% (p<0.01) of baseline at 60 min (FIG. 3G). Mice with deletion of A1 receptors responded less to acupuncture, and the decrease in the amplitude of eEPSP did at no point during the recordings differ significantly from controls mice not exposed to acupuncture. Combined, these observations provide direct evidence for a role of adenosine in the acupuncture-mediated tolerance to pain in models of inflammatory and neuropathic pain. The relative slow time course of fEPSP depression compared with the sharp decrease in eEPSP amplitude following CCPA injection suggest that that adenosine only slowly accumulated in the extracellular space during acupuncture (FIG. 2F). In addition, since the A1R−/− mice experienced some clinical benefits of acupuncture, pathways other than A1 receptors may contribute to acupuncture-mediated pain suppression. These mechanisms could include direct actions of ATP on P2X receptors as proposed in a recent publication, or central release of opioid peptides (Burnstock, G., “Acupuncture: A Novel Hypothesis for the Involvement of Purinergic Signalling,” Med. Hypotheses 73:470-472 (2009); Han, J. S., “Acupuncture and Endorphins,” Neurosci. Lett. 361:258-261 (2004); Zhao, Z. Q., “Neural Mechanism Underlying Acupuncture Analgesia,” Prog. Neurobiol. 85:355-375 (2008), which are hereby incorporated by reference in their entirety). Of note, the peak increases in extracellular adenosine concentrations detected during acupuncture were modest, and thereby primarily acting on anti-nociceptive A1 receptors (Kd ˜20-70 nM). The lack of the pronociceptive, pain facilitatory effects of A2a and A3 receptor activation (except for the immediate pain felt during the needle insertion) can be ascribed to the lower affinity of these receptors (150-300 nM) or that AMP directly activated A1 receptors (Burnstock et al., “The Classification of Receptors for Adenosine and Adenine Nucleotides,” In Methods in Pharmacology, D. Paton, ed. (Plenum Publishing Corporation), pp. 193-212 (1985); Moody et al., “Stimulation of Pl-purinoceptors by ATP Depends Partly on its Conversion to AMP and Adenosine and Partly on Direct Action,” Eur. J. Pharmacol. 97:47-54 (1984), which are hereby incorporated by reference in their entirety). Mice with deletion of A2a receptors exhibited no benefit of either CCPA injection or acupuncture (FIG. 6) compared to WT controls (FIGS. 2 and 3) (Chen et al., “A(2A) Adenosine Receptor Deficiency Attenuates Brain Injury Induced by Transient Focal Ischemia in Mice,” J. Neurosci. 19:9192-9200 (1999), which is hereby incorporated by reference in its entirety).

Accumulation of nucleotides in the interstitial space during acupuncture is, similar to other types of tissue injury, likely a consequence of unspecific membrane damage or opening of stress-activated channels, (Abbracchio et al., “Purinergic Signalling in the Nervous System: An Overview,” Trends Neurosci. 32:19-29 (2009); Sabirov et al., “The Maxi-Anion Channel: A Classical Channel Playing Novel Roles Through an Unidentified Molecular Entity,” J. Physiol. Sci. 59:3-21 (2009), which are hereby incorporated by reference in their entirety). Based on the HPLC analysis of purines in samples of the interstitial fluid, it was speculated that the long-lasting accumulation of AMP in the extracellular space (FIG. 1C) acted as a reservoir for continued generation of adenosine in concentrations sufficient to activate high affinity A1 receptors (Km ˜20-70 nM), but undetectable by HPLC after termination of the acupuncture (Fredholm, B. B., “Adenosine, an Endogenous Distress Signal, Modulates Tissue Damage and Repair,” Cell Death Differ. 14:1315-1323 (2007), which is hereby incorporated by reference in its entirety). Alternatively, AMP may directly activate A1 receptors as previously reported (Burnstock et al., “The Classification of Receptors for Adenosine and Adenine Nucleotides,” In Methods in Pharmacology, D. Paton, ed. (Plenum Publishing Corporation), pp. 193-212 (1985); Moody et al., “Stimulation of P1-purinoceptors by ATP Depends Partly on its Conversion to AMP and Adenosine and Partly on Direct Action,” Eur. J. Pharmacol. 97:47-54 (1984), which are hereby incorporated by reference in their entirety). In either case, the prolonged increase in the extracellular concentration of AMP may represent a key to understand why the anti-analgesic effect of acupuncture outlasts the acupuncture. The relative high concentration of AMP compared to ATP during acupuncture (FIG. 1C) is likely reflects the rapid enzymatic degradation of ATP by ectonucleotidases, since ATP is present in the cytosol of skeletal muscles, fibroblast, fat cells in a concentration of 4-8 mM, or about 100-fold higher than AMP (Poortmans, J., “Principles of Exercise Biochemistry,” Vol 46, 3rd Ed. (Brussels) (2003), which is hereby incorporated by reference in its entirety). The primary ectonucleotidase in peripheral tissue is CD39, which converts ATP to AMP (Cunha et al., “Extracellular Metabolism of Adenine Nucleotides and Adenosine in the Innervated Skeletal Muscle of the Rrog,” Eur. J. Pharmacol. 197:83-92 (1991); Cunha et al., “Extracellular Metabolism of Adenine Nucleotides and Adenosine in the Innervated Skeletal Muscle of the Rrog,” Eur. J. Pharmacol. 197:83-92 (1991); Delgado et al., “T-Tubule Membranes from Chicken Skeletal Muscle Possess an Enzymic Cascade for Degradation of Extracellular ATP,” Biochem. J. 327(Pt 3):899-907 (1997), which are hereby incorporated by reference in their entirety). Using sections of tissue (muscle and subcutis) harvested at the Zusanli point, it was found that phosphate was generated by a rate of 0.428±0.046 μM/mg/min, when 1 mM ATP was added as substrate, whereas addition of AMP (1 mM) produced phosphate by a rate of 0.043±0.005 μM/mg/min (n=3). The slow kinetic of the latter reaction indicate that AMP dephosphorylation is the rate-limiting step in production of adenosine, explaining why the increase in extracellular AMP outlasted the elevations of ATP, ADP, and adenosine (FIG. 1C). AMP is, however, not necessarily degraded to adenosine, because AMP in skeletal muscles also can be deaminated to IMP (Cunha et al., “Extracellular Metabolism of Adenine Nucleotides and Adenosine in the Innervated Skeletal Muscle of the Rrog,” Eur. J. Pharmacol. 197:83-92 (1991), which is hereby incorporated by reference in its entirety) (FIG. 4A). In fact, HPLC analysis indicated that IMP was generated ˜4-fold faster than adenosine in accordance with the high activity of AMP deaminase in skeletal muscles (Cunha et al., “Extracellular Metabolism of Adenine Nucleotides and Adenosine in the Innervated Skeletal Muscle of the Rrog,” Eur. J. Pharmacol. 197:83-92 (1991), which is hereby incorporated by reference in its entirety (FIG. 4B)). 5′-ectonucleotidase, or CD73, is in many tissues the primary enzyme that dephosphorylates AMP. However, it was found that CD73 expression was low in subcutis and below detection in the underlying muscle (FIG. 4C). A CD73 inhibitor, AOPCP, did not reduce dephosphorylation of AMP in sections prepared from tissue close to the Zusanli point (FIG. 4C). In a search for alternative pathways, it was confirmed that prostatic acid phosphatase (PAP) a phosphatase that catalyzes dephosphorylation of AMP was expressed by skeletal muscles (Quintero et al., “Prostatic Acid Phosphatase is not a Prostate Specific Target,” Cancer Res. 67:6549-6554 (2007), which is hereby incorporated by reference in its entirety) (FIG. 4C). An inhibitor of PAP, molybdate partly suppressed dephosphorylation of AMP, suggesting that other (unidentified) phosphatases, in addition to PAP, are involved in conversion of AMP to adenosine in skeletal muscles/subcutis (FIG. 4C).

### Example 8

### Prolonging the Anti-Nociceptive Effect of Acupuncture by Inhibition of AMP Deaminase

AMP deaminase functions as an enzymatic shuttle for degradation of AMP that bypasses adenosine production. Based on the observation that ˜80% of exogenous added AMP was deaminated to IMP and only 20% dephosphorylated to adenosine (FIG. 4B), it was asked whether it is possible to prolong the anti-nociceptive effect of acupuncture by inhibiting AMP deaminase. The effect of the AMPD inhibitor deoxycoformycin in isolated preparations of muscle/subcutis was first tested. Deoxycoformycin reduced AMP conversion to IMP by approximately 50% in isolated muscle/subcutis slices (FIG. 4D). Of note, deoxycoformycin (Pentostatin) is a nucleoside analogue produced by Streptomyces antibioticus, which inhibits DNA synthesis and already is approved by the FDA for treatment a leukemia (Lamanna et al., “Pentostatin Treatment Combinations in Chronic Lymphocytic Leukemia,” Clin. Adv. Hematol. Oncol. 7:386-392 (2009), which is hereby incorporated by reference in its entirety). To evaluate the potential clinical benefit of deoxycoformycin as an adjuvant to acupuncture, deoxycoformycin was administered to mice with either inflammatory or neurogenic pain. The duration by which acupuncture reduced pain in mice that received deoxycoformycin versus vehicle (PBS) was then compared. The 30 min acupuncture session (needle rotated twice every 5 min for a total of 30 min) reduced pain for a duration of ˜1.5 hrs in mice that received vehicle (FIG. 4E-H). Strikingly, mice pretreated with deoxycoformycin exhibited a significant increase in the duration of pain relief. Mechanical allodynia and thermal was suppressed for ˜3.0 hrs in mice suffering from either inflammatory or neurogenic pain treated with a combination of deoxycoformycin and acupuncture (FIG. 4E-H). Of note, deoxycoformycin had no effect on either the tactile or thermal sensitivity when not combined with acupuncture (FIG. 7). These data indicate that suppression of AMP deaminase activity can be used as an adjuvant to acupuncture, which effectively increase its clinical benefits.

Although acupuncture has been practiced for more than 4000 years, it has proven difficult to establish its biological basis (Cabyoglu et al., “The Mechanism of Acupuncture and Clinical Applications,” Int. J. Neurosci. 116:115-125 (2006), which is hereby incorporated by reference in its entirety). The findings reported here, which position adenosine centrally in the mechanistic actions of acupuncture may in retrospect not be surprising. As shown in FIG. 1, insertion and manual rotations of the acupuncture needles triggered a general increase in the extracellular concentration of purines. This was not an unexpected finding, since tissue damage previously has been linked to elevation of nucleotides and adenosine in the extracellular space (Dunwiddie et al., “The Role and Regulation of Adenosine in the Central Nervous System,” Annu. Rev. Neurosci. 24:31-55 (2001); Fredholm, B. B., “Adenosine, an Endogenous Distress Signal, Modulates Tissue Damage and Repair,” Cell Death Differ. 14:1315-1323 (2007); Kerkweg et al., “ATP-Induced Calcium Increase as a Potential First Signal in Mechanical Tissue Trauma. A Laser Scanning Microscopic Study on Cultured Mouse Skeletal Myocytes,” Shock 24:440-446 (2005), which are hereby incorporated by reference in their entirety). Moreover, the anti-nociceptive effects of peripheral, spinal, and supraspinal adenosine A1 receptors is well established (Sawynok, J., “Adenosine Receptor Activation and Nociception,” Eur. J. Pharmacol. 347:1-11 (1998), which is hereby incorporated by reference in its entirety), and herein confirms that peripheral injection of an A1 receptor agonist suppresses hyperalgesia (Karlsten et al., “Local Antinociceptive and Hyperalgesic Effects in the Formalin Test After Peripheral Administration of Adenosine Analogues in Mice,” Pharmacol. Toxicol. 70:434-438 (1992), which is hereby incorporated by reference in its entirety) (FIG. 2). It has previously been shown that local injection of an adenosine kinase inhibitor, which increases the extracellular concentration of adenosine reduces inflammatory pain. Moreover, the same study reported that co-administration of deoxycoformycin augmented the pain relief (Sawynok et al., “Peripheral Antinociceptive Effect of an Adenosine Kinase Inhibitor, With Augmentation by an Adenosine Deaminase Inhibitor, in the Rat Formalin Test,” Pain 74:75-81 (1998), which is hereby incorporated by reference in its entirety). As most other transmitters, adenosine has a short lifespan in the extracellular space due to facilitated uptake by equilibrative nucleoside transporters (Cunha et al., “Extracellular Metabolism of Adenine Nucleotides and Adenosine in the Innervated Skeletal Muscle of the Rrog,” Eur. J. Pharmacol. 197:83-92 (1991), which is hereby incorporated by reference in its entirety). After reuptake, adenosine is quickly converted to AMP by adenosine kinase (Km˜20 nM) assisting the rapid clearance of extracellular adenosine. The observation that AMP remained elevated for hours after acupuncture may therefore represent a key to understand how acupuncture can alleviate pain. The long-lasting increase in AMP following acupuncture likely act as a reservoir for persistent generation of adenosine, or alternatively, AMP may directly activate A1 receptors (Burnstock et al., “The Classification of Receptors for Adenosine and Adenine Nucleotides,” In Methods in Pharmacology, D. Paton, ed. (Plenum Publishing Corporation), pp. 193-212 (1985); Moody et al., “Stimulation of P1-purinoceptors by ATP Depends Partly on its Conversion to AMP and Adenosine and Partly on Direct Action,” Eur. J. Pharmacol. 97:47-54 (1984), which are hereby incorporated by reference in their entirety).

It is tempting to speculate that chiropractic treatment of chronic pain, as well as massage, which basic principle involves mechanical manipulation of joints and muscles, also are associated with efflux of cytosolic ATP resulting in a rise in the extracellular concentration of adenosine. Adenosine may accumulate during both of these treatments, and similarly to acupuncture, dampen pain by activation of A1 receptors on sensory afferents or ascending nerve tracks. Moreover, acupuncture is also frequently used in the treatment of diseases with an inflammatory component, such as arthritis and tendinitis. It is in this regard of interest that the anti-inflammatory properties of adenosine are well-established Kavoussi et al., “The Neuroimmune Basis of Anti-Inflammatory Acupuncture,” Integr. Cancer Ther. 6:251-257 (2007); Lee et al., “Acupuncture for Rheumatoid Arthritis: A Systematic Review,” Rheumatology (Oxford) 47:1747-1753 (2008); Zhang et al., “Electroacupuncture Attenuates Inflammation in a Rat Model,” J. Altern. Complement Med. 11:135-142 (2005), which are hereby incorporated by reference in their entirety).

In summary, this is the first study, that link the analgesic action of acupuncture treatment to release of adenosine and activation of A1 receptors on ascending nerves. The most important practical aspect of the present invention is the observation that pharmacologic manipulations of AMP degradation prolonged the analgesic effect of acupuncture. Medication that interferes with AMP metabolism may thereby have the potential to improve the clinical benefits of acupuncture.

### Example 9

### cAMP and PKA Inhibition Effects in Mice

Additional data shows that acupuncture mediated A1 receptor activation mediates its effect through the cAMP and PKA pathway. Earlier studies have described that neuropathic pain is associated with a sustained increase in the intracellular concentration of cAMP in DRG neurons (Aley et al., “Role of Protein Kinase A in the Maintenance of Inflammatory Pain,” J Neurosci 19:2181-2186 (1999); Zheng et al., “Dissociation of Dorsal Root Ganglion Neurons Induces Hyperexcitability That is Maintained by Increased Responsiveness to cAMP and cGMP,” J Neurophysiol 97:15-25 (2007), which are hereby incorporated by reference in their entirety).

Consistent with this, data in FIG. 8 illustrates that when the membrane permeable cAMP (dibutyryl cAMP) was injected to the Zusanli point, sensitivity to touch increased transiently in WT mice. Activation of A1 receptors inhibits adenylate cyclase and thereby reduces the levels of cAMP (Elzein et al., “A1 Adenosine Receptor Agonists and Their Potential Therapeutic Applications,” Expert Opin Investig Drugs 17:1901-1910 (2008), which is hereby incorporated by reference in its entirety). Thus, acupuncture-induced activation of A1R may decrease cAMP levels which, in turn, inhibits the release of proinflammatory neuropetides (substance P and CGRP) and hence, a suppression of hyperexcitability of nociceptive pathways (Neumann et al., “Inflammatory Pain Hypersensitivity Mediated by Phenotypic Switch in Myelinated Primary Sensory Neurons,” Nature 384:360-364 (1996), which is hereby incorporated by reference in its entirety).

Additional data in FIG. 9 supports the role of cAMP and PKA in pain sensitization at acupoints. The experiments were designed to test the hypothesis that this pain pathway is modified by acupuncture. The PKA inhibitor H-89 reduced pain temporarily when injected into the Zusanli point in mice with chronic pain induced by CFA injection.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of improving the therapeutic effect of acupuncture in a subject, said method comprising:
administering adenosine, an adenosine mimetic, an adenosine modulator, an adenosine transport inhibitor, enzymes involved in adenosine metabolism, and/or an adenosine receptor agonist to the subject under conditions effective to improve the therapeutic effect of the acupuncture.

2. The method of claim 1, wherein the therapeutic effect of the acupuncture is selected from the group consisting of pain relief and treatment of an inflammatory condition.

3. The method of claim 2, wherein the therapeutic effect of acupuncture is pain relief.

4. The method of claim 2, wherein the therapeutic effect of acupuncture is treatment of an inflammatory condition.

5. The method of claim 4, where the inflammatory condition is selected from the group consisting of arthritis, and tendinitis.

6. The method of claim 1, wherein said administering involves administration of a protein.

7. The method of claim 1, wherein said administering involves administration of a nucleic acid.

8. The method of claim 7, wherein the nucleic acid is in a viral vector.

9. The method of claim 1, wherein said administering involves administration of a small molecule.

10. The method of claim 1, wherein adenosine is administered.

11. The method of claim 1, wherein an adenosine receptor agonist is administered, said adenosine receptor agonist being selected from the group consisting of adenosine receptor congeners, N6-cyclopentyladenosine, N6-cyclohexyladenosine, 2-chloro-cyclopentyladenosine, N-(3(R))-tetrahydrofuranyl)-6-aminopurine riboside, nucleoside transporters, and combinations thereof.

12. The method of claim 1, wherein an adenosine transport inhibitor is administered, said adenosine transport inhibitor being selected from the group consisting of dipyridamole, nitrobenzylthioinosine, dilazep, lidoflazines, benzodiazepines, dihydropyridies, xanthine, quinoline derivatives, and combinations thereof.

13. The method of claim 1, wherein an enzyme involved in adenosine metabolism is administered, said enzyme involved in adenosine metabolism being selected from the group consisting of ecto-5′-nucleotidase modulator, S-adenosylhomocysteine hydrolase inhibitor, adenosine diaminase inhibitor, and combinations thereof.

14. The method of claim 1, wherein said administering is systemic.

15. The method of claim 1 further comprising:
selecting a subject in need of acupuncture therapy, wherein the selected subject is subjected to said administering.

16. The method of claim 1, wherein Tecadenoson, CVT-3619, BAY-68-4986, INFO-8875, and/or DTI-009 are administered to the subject.

17. The method of claim 13, wherein an ecto-5′-nucleotidase modulator is administered, said ecto-5′-nucleotidase modulator being selected from the group consisting of thiamine monophosphatase (TMPase), prostatic acid monophosphatase (PAP), and transmembrane isoform of PAP (TM-PAP).

18. The method of claim 17, wherein the ecto-5′-nucleotidase modulator is PAP.

19. The method of claim 18, wherein the PAP is administered orally, subcutaneously, intravenously, intramuscularly, intraperitoneally, by intransal instillation, or by application to mucous membranes.

20. The method of claim 18, wherein the PAP is administered parenterally.
